(19) United States
(12) Patent Application Publication
(10) Pub. No.: US 2024/0237997 A1
(43) Pub. Date: Jul. 18, 2024

(54) THROMBUS REMOVAL SYSTEMS AND ASSOCIATED METHODS

(71) Applicant: Shifamed Holdings, LLC, Campbell, CA (US)

(72) Inventors: Amr SALAHIEH, Saratoga, CA (US); Aadel AL-JADDA, San Carlos, CA (US); Kevin MULLER, San Carlos, CA (US)

(21) Appl. No.: 18/688,941

(22) PCT Filed: Sep. 6, 2022
(86) PCT No.: PCT/US2022/075986

§ 371 Date: Mar. 4, 2024

Related U.S. Application Data
(60) Provisional application No. 63/240,829, filed on Sep. 3, 2021.

(51) Int. Cl. A61B 17/22; A61B 17/00

(52) U.S. Cl. CPC A61B 17/22; A61B 17/00234; A61B 2017/00238; A61B 2017/00305; A61B 2017/00318; A61B 2017/00367; A61B 2017/22045; A61B 2017/22079

(57) ABSTRACT

The present technology relates to systems and methods for delivering a dilator sheath to a target location within a patient. The dilator sheath can include a guidewire lumen configured to receive a guidewire. Embodiments are provided in which an interference fit between the guidewire and the guidewire lumen facilitate steering of the dilator sheath in at least one direction. Methods of delivering the dilator sheath are provided. Methods of delivering a medical device with the dilator sheath are also provided.

### CROSS REFERENCE TO RELATED APPLICATIONS

This application claims the benefit of U.S. Application No. 63/240,829, filed Sep. 3, 2021, the disclosure of which is herein incorporated by reference in its entirety.

### RELATED APPLICATIONS

This application is related to International Application No. PCT/US2021/020915, filed Mar. 4, 2021, and International Application No. PCT/US2022/033024, filed Jun. 10, 2022, the disclosures of which are incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present technology generally relates to medical devices and, in particular, to systems including aspiration and fluid delivery mechanisms and associated methods for removing a thrombus from a mammalian blood vessel.

### BACKGROUND

Thrombotic material may lead to a blockage in fluid flow within the vasculature of a mammal. Such blockages may occur in varied regions within the body, such as within the pulmonary system, peripheral vasculature, deep vasculature, or brain. Pulmonary embolisms typically arise when a thrombus originating from another part of the body (e.g., a vein in the pelvis or leg) becomes dislodged and travels to the lungs. Anticoagulation therapy is the current standard of care for treating pulmonary embolisms, but may not be effective in some patients. Additionally, conventional devices for removing thrombotic material may not be capable of navigating the vascular anatomy of the lungs, may not be effective in removing thrombotic material, and/or may lack the ability to provide sensor data or other feedback to the clinician during the thrombectomy procedure.

### SUMMARY OF THE DISCLOSURE

A method for steering an introducer catheter is provided, the method comprising introducing a guidewire into a lumen, the guidewire including an interference feature on the guidewire, advancing the guidewire towards a target location in the lumen, inserting the guidewire into a guidewire lumen of the introducer catheter, wherein the guidewire lumen of the introducer catheter has a smaller diameter than the interference feature of the guidewire, advancing the introducer catheter along the guidewire, and engaging the interference feature of the guidewire against the guidewire lumen of the introducer catheter to steer the introducer catheter.

In some embodiments, the interference feature is disposed on a distal portion of the guidewire.

In other embodiments, the interference feature comprises a step-up portion in which a diameter of the guidewire increases to be larger than a guidewire lumen diameter of the introducer catheter.

In some embodiments, the interference feature comprises a protrusion having a larger diameter than a guidewire lumen diameter of the introducer catheter.

In one example, the method includes delivering a medical device to the target location via a lumen of the introducer catheter.

In some embodiments, the lumen comprises a blood vessel. In one example, the target location comprises a target thrombus location. Additionally, the medical device comprises a thrombus removal device.

In some examples, the interference feature comprises an expandable member, wherein engaging the interference feature further comprises expanding the expandable member.

In some embodiments, engaging the interference feature further comprises pulling the guidewire proximally to engage the interference feature with the guidewire lumen.

The method of claim 1, wherein engaging the interference feature further comprises pushing the guidewire distally to engage the interference feature with the guidewire lumen.

An introducer catheter device, comprising an elongate shaft having a distal portion, a device lumen configured to receive a medical device, a guidewire lumen having a first diameter, and a guidewire configured to be disposed within the guidewire lumen, the guidewire having a second diameter smaller than the first diameter, the guidewire also having at least one interference feature having a third diameter larger than the first diameter, the interference feature being configured to create an interference fit with the guidewire lumen when the guidewire is moved relative to the guidewire lumen for steering of the introducer catheter device in at least one direction.

In some embodiments, the distal portion includes a dilator portion configured to stretch or expand a tissue.

In one implementation, the guidewire lumen extends along only the distal portion. In other embodiments, the guidewire lumen extends along the distal portion and the elongate shaft.

In some examples, the interference feature comprises a step-up portion that includes a transition from the second diameter to the third diameter. In other embodiments, the interference feature comprises a protrusion.

In one embodiment, the interference feature comprises a spherical shape.

In some examples, the interference feature comprises an expandable structure.

In one embodiment, the interference feature comprises a balloon.

In some examples, the interference feature is positioned distal to the guidewire lumen when the guidewire is disposed within the guidewire lumen. In one embodiment, the interference feature is configured to provide an interference fit with the guidewire lumen when the guidewire is pulled proximally with respect to the introducer catheter.

In some embodiments, the interference feature is positioned proximal to the guidewire lumen when the guidewire is disposed within the guidewire lumen. In one implementation, the interference feature is configured to provide an interference fit with the guidewire lumen when the guidewire is pushed distally with respect to the introducer catheter.

In some examples, the guidewire further comprises a second interference feature positioned proximal to the guidewire lumen when the guidewire is disposed within the guidewire lumen. In this example, the second interference feature is configured to provide an interference fit with the guidewire lumen when the guidewire is pushed distally with respect to the introducer catheter.

An introducer catheter device is provided, comprising an elongate shaft having a distal portion, a device lumen configured to receive a medical device, a guidewire lumen having a depressible section, and a guidewire configured to be disposed within the guidewire lumen, wherein the depressible section is configured to expand to create an interference fit with the guidewire lumen to facilitate steering of the introducer catheter device in at least two directions.

In some embodiments, the introducer catheter is configured to be steered in at least two directions by pulling or pushing against the guidewire when the depressible section is expanded.

In one example, the distal portion includes a dilator portion configured to stretch or expand a tissue.

In another embodiment, the guidewire lumen extends along only the distal portion.

In some embodiments, the guidewire lumen extends along the distal portion and the elongate shaft.

In one example, the depressible section further comprises an inflatable member.

In another example, the inflatable member comprises a balloon fluidly coupled to an inflation lumen.

An introducer catheter device is provided, comprising an elongate shaft having a distal portion, a device lumen configured to receive a medical device, a guidewire lumen, and a guidewire configured to be disposed within the guidewire lumen, the guidewire having a depressible section configured to expand to create an interference fit with the guidewire lumen to facilitate steering of the introducer catheter device in at least two directions.

In some embodiments, the introducer catheter is configured to be steered in at least two directions by pulling or pushing against the guidewire when the depressible section is expanded.

In one example, the distal portion includes a dilator portion configured to stretch or expand a tissue.

In another embodiment, the guidewire lumen extends along only the distal portion.

In some embodiments, the guidewire lumen extends along the distal portion and the elongate shaft.

In one example, the depressible section further comprises an inflatable member.

In another example, the inflatable member comprises a balloon fluidly coupled to an inflation lumen.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1-1L illustrate various views of a portion of a thrombus removal system including a distal portion of an elongated catheter configured in accordance with an embodiment of the present technology.

FIGS. 2A-2D illustrates plan views of various configurations of irrigation ports and fluid streams of a thrombus removal system according to embodiments of the present technology.

FIGS. 3A-3H illustrate an elevation view of various configurations of irrigation ports of a thrombus removal system according to embodiments of the present technology.

FIGS. 4A-4H illustrate an elevation view of various configurations of irrigation ports and fluid streams of a thrombus removal system according to embodiments of the present technology.

FIGS. 5A-5G illustrate various configurations of irrigation ports of a thrombus removal system according to embodiments of the present technology.

FIGS. 6A-6C illustrate various embodiments of a thrombus removal system including a saline source, an aspiration system, and one or more controls for controlling irrigation and/or aspiration of the system.

FIGS. 7A-7D show one embodiment of a dilator sheath device.

FIGS. 8A-8D show another embodiment of a dilator sheath device.

FIGS. 9A-9C show the dilator sheath device with a medical device extended distally from the device.

FIGS. 10A-10B show another embodiment of a dilator sheath device.

FIGS. 11A-11C show another embodiment of a dilator sheath device.

FIG. 12 is a flowchart that describes a method of using a sheath device.

### DETAILED DESCRIPTION

This application is related to disclosure in International Application No. PCT/US2021/020915, filed Mar. 4, 2021, the disclosure of which is incorporated by reference herein for all purposes.

The present technology is generally directed to thrombus removal systems and associated methods. A system configured in accordance with an embodiment of the present technology can include, for example, an elongated catheter having a distal portion configured to be positioned within a blood vessel of the patient, a proximal portion configured to be external to the patient, a fluid delivery mechanism configured to fragment the thrombus with pressurized fluid, an aspiration mechanism configured to aspirate the fragments of the thrombus, and one or more lumens extending at least partially from the proximal portion to the distal portion.

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to the figures.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

Although some embodiments herein are described in terms of thrombus removal, it will be appreciated that the present technology can be used and/or modified to remove other types of emboli that may occlude a blood vessel, such as fat, tissue, or a foreign substance. Additionally, although some embodiments herein are described in the context of thrombus removal from a pulmonary artery (e.g., pulmonary embolectomy), the technology may be applied to removal of thrombi and/or emboli from other portions of the vasculature (e.g., in neurovascular, coronary, or peripheral applications). Moreover, although some embodiments are discussed in terms of maceration of a thrombus with a fluid, the present technology can be adapted for use with other techniques for breaking up a thrombus into smaller fragments or particles (e.g., ultrasonic, mechanical, enzymatic, etc.)

The headings provided herein are for convenience only and do not interpret the scope or meaning of the claimed present technology.

### Systems for Thrombus Removal

As provided above, the present technology is generally directed to thrombus removal systems. Such systems include an elongated catheter having a distal portion positionable within a blood vessel of the patient (e.g., an artery or vein), a proximal portion positionable outside the patient's body, a fluid delivery mechanism configured to fragment the thrombus with pressurized fluid, an aspiration mechanism configured to aspirate the fragments of the thrombus, and one or more lumens extending at least partially from the proximal portion to the distal portion. The fluid delivery mechanism and aspiration mechanism can be any as are described in embodiments of the Appendix. In some embodiments, the systems herein are configured to engage a thrombus in a patient's blood vessel, break the thrombus into small fragments, and aspirate the fragments out of the patient's body. The pressurized fluid streams (e.g., jets) function to cut or macerate thrombus, before, during, and/or after at least a portion of the thrombus has entered the aspiration lumen or a funnel of the system. Fragmentation helps to prevent clogging of the aspiration lumen and allows the thrombus removal system to macerate large, firm clots that otherwise could not be aspirated. As used herein, “thrombus” and “embolism” are used somewhat interchangeably in various respects. It should be appreciated that while the description may refer to removal of “thrombus,” this should be understood to encompass removal of thrombus fragments and other emboli as provided herein.

According to embodiments of the present technology, a fluid delivery mechanism can provide a plurality of fluid streams (e.g., jets) to fluid apertures of the thrombus removal system for macerating, cutting, fragmenting, pulverizing and/or urging thrombus to be removed from a proximal portion of the thrombus removal system. The thrombus removal system can include an aspiration lumen extending at least partially from the proximal portion to the distal portion of the thrombus removal system that is adapted for fluid communication with an aspiration pump (e.g., vacuum source). In operation, the aspiration pump may generate a volume of lower pressure within the aspiration lumen near the proximal portion of the thrombus removal system, urging aspiration of thrombus from the distal portion.

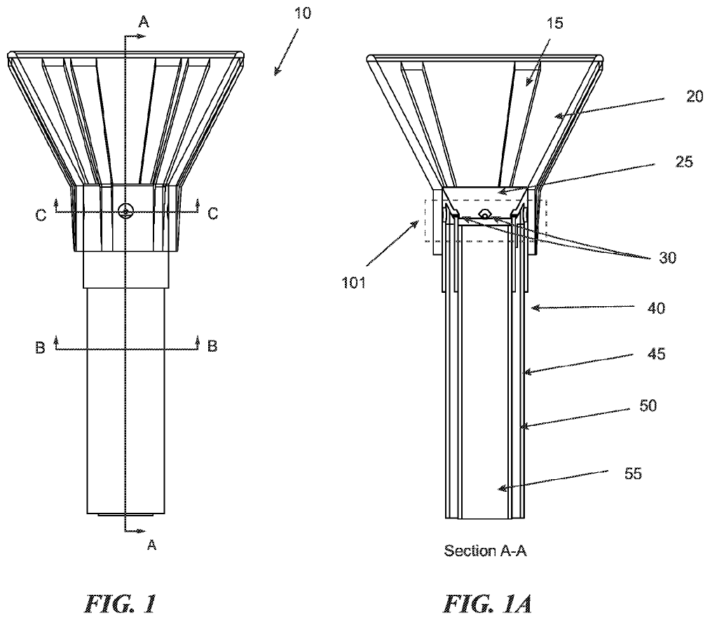

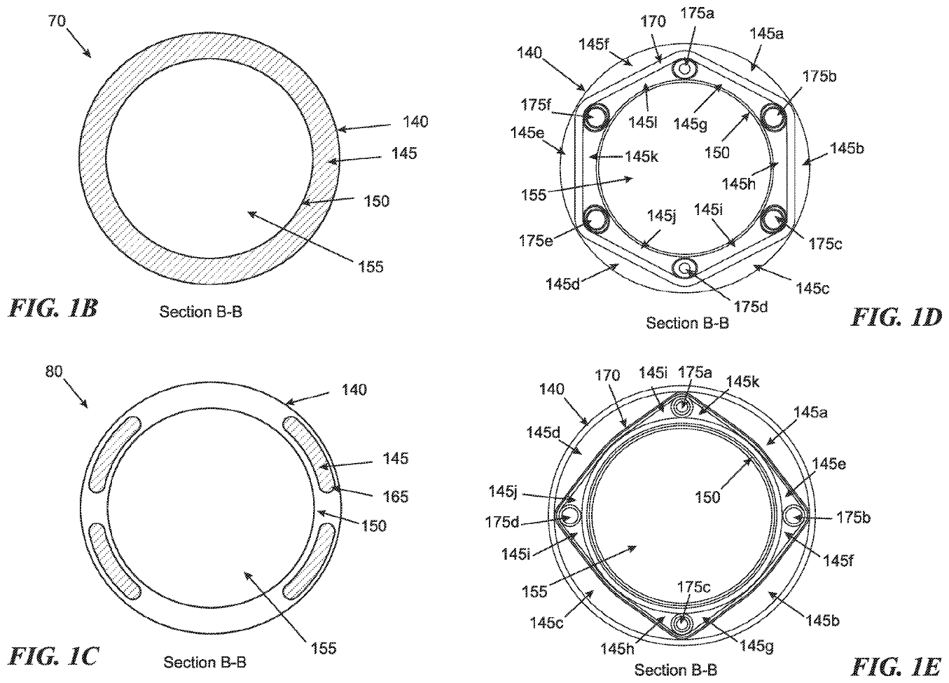

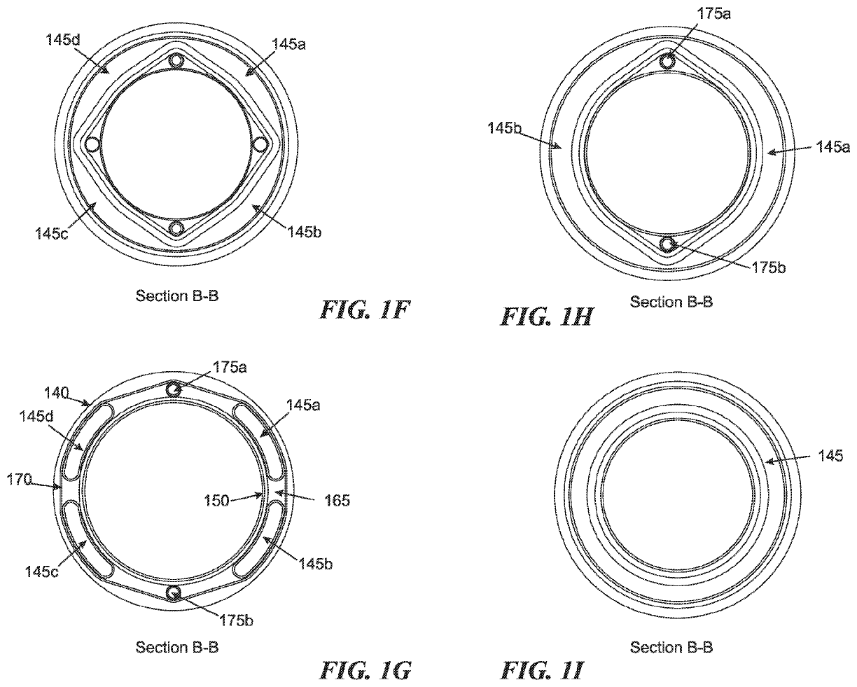

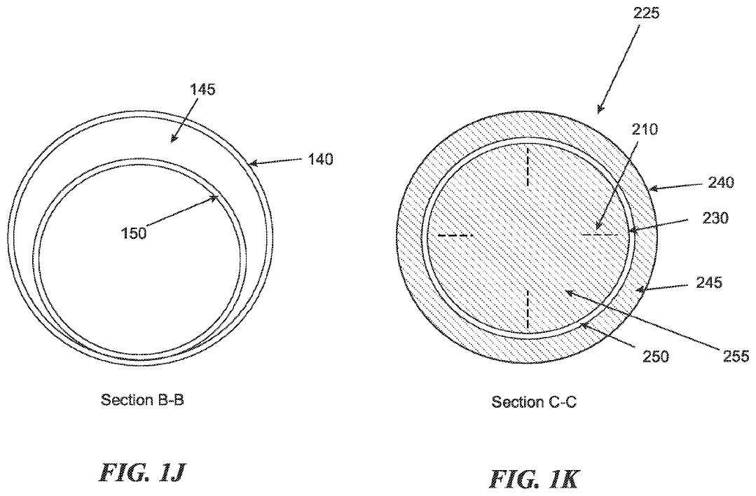

FIG. 1 illustrates a distal portion 10 of a thrombus removal system according to an embodiment of the present technology. FIG. 1A Section A-A illustrates an elevation sectional view of the distal portion. The example section A-A in FIG. 1A depicts a funnel 20 that is positioned at the distal end of the distal portion 10, the funnel adapted to engage with thrombus and/or a tissue (e.g., vessel) wall to aid in thrombus fragmentation and/or removal. The funnel can be formed according to any of the constructions described in the Appendix. The example section A-A in FIG. 1A depicts a double walled thrombus removal device construction having an outer wall/tube 40 and an inner wall/tube 50. An aspiration lumen 55 is formed by the inner wall 50 and is centrally located. A generally annular volume forms at least one fluid lumen 45 between the outer wall 40 and the inner wall 50. The fluid lumen 45 is adapted for fluid communication with the fluid delivery mechanism. One or more apertures (e.g., nozzles, orifices, or ports) 30 are positioned in the thrombus removal system to be in fluid communication with the fluid lumen 45 and an irrigation manifold 25. In operation, the ports 30 are adapted to direct (e.g., pressurized) fluid toward thrombus that is engaged with the distal portion 10 of the thrombus removal system.

In various embodiments, the system can have an average flow velocity within the fluid lumen of up to 20 m/s to achieve consistent and successful aspiration of clots. In some embodiments, the fluid source itself can be delivered in a pulsed sequence or a preprogrammed sequence that includes some combination of pulsatile flow and constant flow to deliver fluid to the jets. In these embodiments, while the average pulsed fluid velocity may be up to 20 m/s, the peak fluid velocity in the lumen may be up to 30 m/s or more during the pulsing of the fluid source. In some embodiments, the jets or apertures are no smaller than 0.0100″ or even as small as 0.008″ to avoid undesirable spraying of fluid. In some embodiments, the system can have a minimum vacuum or aspiration pressure of 1-3 inHg, to remove target clots after they have been macerated or broken up with the jets described above.

The thrombus removal system can be sized and configured to access and remove thrombi in various locations or vessels within a patient's body. It should be understood that while the dimensions of the system may vary depending on the target location, generally the same features and components described herein will be implemented in the thrombus removal system regardless of the application. For example, a thrombus removal system configured to remove pulmonary embolism (PE) from a patient may have an outer wall/tube with a size of approximately 11-13 Fr, or preferably 12 Fr, and an inner wall/tube with a size of 7-9 Fr, or preferably 8 Fr. A deep vein thrombosis (DVT) device, on the other hand, may have an outer wall/tube with a size of approximately 9-11 Fr, or preferably 10 Fr, and an inner wall/tube with a size of 6-9 Fr, or preferably 7.5 Fr. Applications are further provided for ischemic stroke and peripheral embolism applications.

Referring to FIG. 1A, the funnel 20 can comprise a compliant material such as a polymer and include a plurality of grooves or slots 15 configured to receive one or more shape memory structures (not shown). In some embodiments, the shape memory structure can comprise a shape memory alloy such as nitinol. In some embodiments, the shape memory structures can be pre-biased to expand outwards, causing the funnel to expand when a constraining member, such as a delivery sheath, is removed from the funnel. In the illustrated example of FIG. 1A, the slots and shape memory structures extend generally along a longitudinal axis of the device (e.g., proximally to distally). In other embodiments, the slots and shape memory materials can be placed radially or in other configurations within the funnel.

Still referring to FIG. 1B, in one embodiment the funnel can extend over an outer wall of the device, as shown. This can ensure that the compliant material of the funnel covers or protects the patient from potentially sharp corners or edges of the outer wall or manifold of the thrombus removal device. In the illustrated embodiment, the funnel is positioned over the manifold, but in other embodiments, the manifold can be integrated with or positioned within the funnel. For example, in one embodiment the funnel can comprise a compliant cone-shaped bladder, and the manifold can be integrated into or positioned within the bladder. This can facilitate jets or apertures within the funnel itself, which can then receive fluid from the fluid lumens via the manifold.

The funnels described herein generally have a closed configuration that is useful for navigation and placement of the device, and an expanded configuration in which the funnel expands to contact and occlude the target vessel during therapy. In some embodiments, the funnel is passively or self-expanded (e.g., via a shape memory material) and in other embodiments the funnel is actively expanded (e.g., via a mechanical actuation). In additional embodiments, the funnel can be deployed via insertion or retraction of the two components of the device about one another. For example, an outer tube of the device could be attached to the funnel, and a concentric tube inside the outer tube could be the outer wall of the thrombus removal device. The outer tube could be moved relative to the inner tube to cause the funnel to expand or retract. In other embodiments, this relative motion to open and close could be done using rotation as well.

Section B-B of FIG. 1B illustrates in plain view a portion of the thrombus removal system that is proximal to the funnel and irrigation manifold. Section B-B depicts an outer wall 140, an inner wall 150, an aspiration lumen 155 and a fluid lumen 145. In some embodiments, in cross-section the aspiration lumen 155 is generally circular and the fluid lumen 145 is generally annular in shape (e.g., cross-section 70). It will be appreciated that alternative constructions and/or arrangements of the inner wall 150 and the outer wall 140 produce variations in cross-sectional shape of the aspiration and fluid lumens 155 and 145. For example, the inner wall 150 can be shaped to form an aspiration lumen 155 that, in cross-section, is generally oval, circular, rectilinear, square, pentagonal, or hexagonal. The inner and outer walls 150 and 140 can be shaped and arranged to form a fluid lumen 145 that, in cross-section, is generally crescent-shaped, diamond shaped, or irregularly shaped. For example, referring to FIG. 1C Section B-B, the region between the inner wall 150 and the outer wall 140 can include one or more wall structures 165 that form respective fluid lumens 145 (e.g., as in cross-section 80). The wall structures 165 can be formed by lamination between the outer and inner walls 140 and 150, or by a multi-lumen extrusion that forms a plurality of the wall structures.

Section B-B of FIGS. 1D-1H illustrate additional examples of a portion of the thrombus removal system that is proximal to the funnel and irrigation manifold Similar to the embodiments described above, the portion in these examples can include an outer wall 140, an inner wall 150, and an aspiration lumen 155. Additionally, the illustrated portion of the thrombus removal system can include a middle wall 170 disposed between the outer wall 140 and the inner wall 150. The middle wall 170 enables further segmentation of the annular space between the inner wall and outer wall into a plurality of distinct fluid lumens and/or auxiliary lumens. For example, referring to FIG. 1D, the middle wall can be generally hexagon shaped, and the annular space can include a plurality of fluid lumens 145a-1451 and a plurality of auxiliary lumens 175a-175f. As shown in FIG. 1D, the fluid lumens can be formed by some combination of the outer wall 140 and the middle wall 170, or between the middle wall 170, the inner wall 150, and two of the auxiliary lumens. For example, fluid lumen 145a is formed in the space between outer wall 140 and middle wall 170. However, fluid lumen 145g is formed in the space between middle wall 170, inner wall 150, auxiliary lumen 175a, and auxiliary lumen 175b. Generally, the fluid lumens are configured to carry a flow of fluid such as saline from a saline source of the system to one or more ports/apertures/orifices of the system. The auxiliary lumens can be configured for a number of functions. In some embodiments, the auxiliary lumens can be coupled to the fluid/saline source and to the apertures to be used as additional fluid lumens. In other embodiments, the auxiliary lumens can be configured as steering ports and can include a guide wire or steering wire within the lumen for steering of the thrombus removal system. Additionally, in other embodiments, the auxiliary lumens can be configured to carry electrical, mechanical, or fluid connections to one or more sensors. For example, the system may include one or more electrical, optical, or fluid based sensors disposed along any length of the system. The auxiliary ports can therefore be used to connect to the sensors, e.g., by electrical connection, optical connection, mechanical/wire connection, and/or fluid connection.

It should be understood that in some embodiments, all the fluid lumens are fluidly connected to all of the jets or apertures of the thrombus removal device. Therefore, when a flow of fluid is delivered from the fluid lumen(s) to the jets, all jets are activated with a jet of fluid at once. However, it should also be understood that in some embodiments, the fluid lumens are separate or distinct, and these distinct fluid lumens may be fluidly coupled to one or more jets but not to all jets of the device. In these embodiments, a subset of the jets can be controlled by delivering fluid only to the fluid lumens that are coupled to that subset of jets. This enables additional functionality in the device, in which specific jets can be activated in a user defined or predetermined order. In embodiments where the jets are disposed on or within the funnel, the fluid lumens can be fluidly coupled to the funnel jets. Jets can be placed at any position within the funnel, such as along the edge of the funnel or in the main body of the funnel. Jets in the funnel can comprise a hole in the wall of the funnel, an extension of the fluid lumen that exits the funnel, or nozzles disposed in the funnel, etc. As mentioned above, this can be achieved by integrating or fluidly coupling the funnel with the manifold or directly to the fluid lumens. In some embodiments, the funnel may include a baffle structure or other systems for controlling fluids.

Section B-B of FIG. 1E illustrates another embodiment of the portion of the thrombus removal system that is proximal to the funnel and irrigation manifold. Similar to the embodiment of FIG. 1D, this embodiment also includes a middle wall 170. However, the middle wall in this example is generally square shaped, facilitating the formation of fluid lumens 145a-145k and auxiliary lumens 175a-175d. The example illustrated in section B-B of FIG. 1F is similar to that of the embodiment of FIG. 1E, however this embodiment includes only fluid lumens 145a-145d. The fluid lumens 145e-145k from the embodiment of FIG. 1E are not used as fluid lumens in this embodiment. They can be, for example, empty lumens, vacuum, filled with an insulative material, and/or filled with a radio-opaque material or any other material that may help visualize the thrombus removal system during therapy. The embodiment 1F includes the same four auxiliary reports as illustrated and described in the embodiment of FIG. 1E.

Section B-B of FIG. 1G illustrates another example of a portion of the thrombus removal system that is proximal to the funnel and irrigation manifold. Similar to the embodiments described above, the illustrated portion of the thrombus removal system can include a middle wall 170 disposed between the outer wall 140 and the inner wall 150. However, this embodiment includes four distinct fluid lumens 145a-145d formed by wall structures 165. As with the embodiment of FIG. 1C, the wall structures 165 can be formed by lamination between the outer and inner walls 140 and 150, or by a multi-lumen extrusion that forms a plurality of the wall structures. As shown, this embodiment can include a pair of auxiliary lumens 175a and 175b, which can be used, for example, for steering or for sensor connections as described above.

Section B-B of FIG. 1H is another similar embodiment in which the middle wall and outer wall can be used to form fluid lumens 145a and 145b. Auxiliary lumens 175a and 175b can be formed in the space between the middle wall and the inner wall. It should be understood that the middle wall can contact the outer wall to create independent fluid lumens 145a and 145b. However, in other embodiments, it should be understood that the middle wall may not contact the outer wall, which would facilitate a single annular fluid lumen, such as is shown by fluid lumen 145 in Section B-B of FIG. 1I. In another embodiment, as shown in Section B-B of FIG. 1J, the inner wall 150 and the outer wall 140 may not be concentric, which facilitates formation of an annular space and/or fluid lumen 145 that is thicker or wider on one side of the device relative to the other side. As shown in FIG. 1J, a distance between the outer wall 140 and inner wall at the top (e.g., 12 o'clock) portion of the device is larger than a distance between the outer wall and inner wall at the bottom (e.g., 6 o'clock) portion of the device.

Section C-C of FIG. 1K illustrates in plain view a portion of the thrombus removal system comprising an irrigation manifold 225. Section C-C depicts an outer wall 240, an inner wall 250, a fluid lumen 245, an aspiration lumen 255, and ports 230 for directing respective fluid streams 210.

Detail View 101 of FIG. 1L illustrates a section view in elevation of a portion of the irrigation manifold 25 that includes a plurality of ports 230 that are formed within an inner wall 250. In some embodiments, a thickness of one or more walls of the thrombus removal system may be varied along its axial length and/or its circumference. As shown in Detail View 101, inner wall 250 has a first thickness 265 in a region 250 that is proximal to the irrigation manifold 25, and a second thickness 270 in a region 235 that includes the ports 230. In some embodiments, the second thickness 270 is greater than the first thickness 265. The first thickness 265 can correspond to a general wall thickness of the inner wall 50 and/or of the outer wall 40, which can be from about 0.10 mm to about 0.60 mm, or any value within the aforementioned range. The second thickness 270 can be from about 0.50 mm to about 0.70 mm, from about 0.70 mm to about 0.90 mm, or from about 0.90 mm to about 1.20 mm. The second thickness 270 can be any value within the aforementioned range. The dimension of the second thickness 270 can be selected to provide a fluid path through the ports 230 that produces a generally laminar flow for a fluid stream that is directed therethrough, when the fluid delivery mechanism supplies fluid via the fluid lumen 245 at a typical operating pressure. Such operating pressure can be from about 10 psi to about 60 psi, from about 60 psi to about 100 psi, or from about 100 psi to about 150 psi. The operating pressure of the fluid delivery mechanism can be any value within the aforementioned range of values. In some embodiments, the fluid delivery mechanism is operated in a high pressure mode, having a pressure from about 150 psi to about 250 psi, from about 250 psi to about 350 psi, from about 350 psi to about 425 psi, or from about 425 psi to about 500 psi. The operating pressure of the fluid delivery mechanism in the high pressure mode can be any value within the aforementioned range of values. Generally, the length of the aperture or hole through the walls that is used to form the ports 230 needs to have a length sufficient to prevent formation of a spray or mist as the fluid exits the ports. Instead, a focused jet or stream is desired. Given the parameters described above, the length of the apertures through the walls that are used to form the ports should be at least 0.25 mm in length, optionally up to 0.4 mm or up to 1 mm or greater in length. Any lengths shorter than that may undesirably lead to mist or spray ejection from the ports, which will not effectively break up or macerate target clots.

In some embodiments, a profile (cross-sectional dimension) of a port 230 varies along its length (e.g., is non-cylindrical). A variation in the cross-sectional dimension of the port may alter and/or adjust a characteristic of fluid flow along the port 230. For example, a reduction in cross-sectional dimension may accelerate a flow of fluid through the port 230 (for a given volume of fluid). In some embodiments, a port 230 may be conical along its length (e.g., tapered), such that its smallest dimension is positioned at the distal end of the port 230, where distal is with respect to a direction of fluid flow.

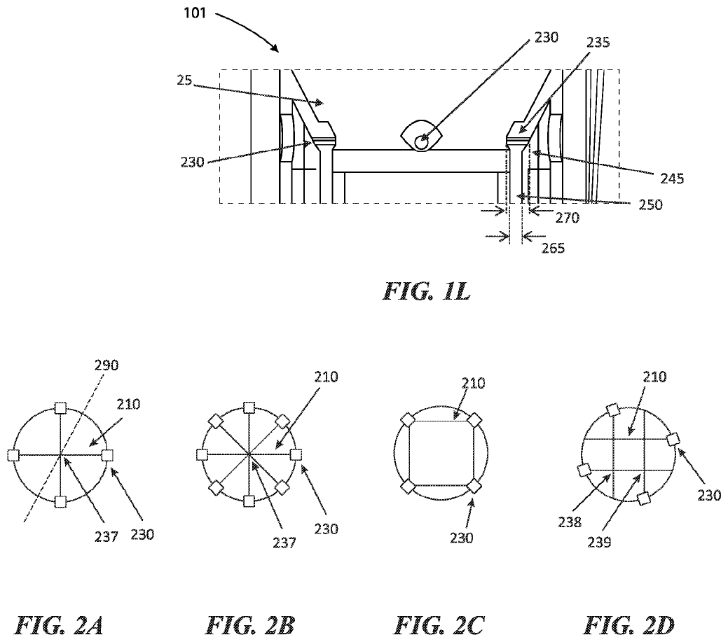

In some embodiments, the port 230 is formed to direct the fluid flow along a selected path. FIGS. 2A-2E illustrate various embodiments of arrangements of ports 230 for directing respective fluid streams 210. In some embodiments, such as those shown in FIGS. 2A and 2B, at least two ports 230 are arranged to produce (e.g., respective) fluid streams 210 that intersect at an intersection region 237 of the thrombus removal system. An intersection region 237 can be a region of increased fluid momentum and/or energy transfer, which increase is with respect to individual fluid streams that are not directed to combine at the intersection. The increased fluid momentum and/or energy transfer at an intersection may advantageously fragment thrombus more efficiently and/or quickly. In some embodiments, an intersection region can be formed from at least 2, at least 3, at least 4, at least 5, at least 6, at least 7, at least 8, at least 9, or at least 10 fluid streams 210. An intersection region can be generally near a central axis 290 of the thrombus removal system (e.g., 237), or away from the central axis (e.g., 238 and 239 in the embodiment of FIG. 2D). In some embodiments, at least two intersection regions (e.g., 238 and 239) are formed. In some embodiments, one or more ports 230 are arranged to direct a fluid stream 210 along an oblique angle with respect to the central axis of the thrombus removal system. An operating pressure of the fluid delivery mechanism may be selected to approach a targeted fluid velocity for a fluid stream 210 that is delivered from a port 230. The targeted fluid velocity for a fluid stream 210 can be about 5 meters/second (m/s), about 8 m/s, about 10 m/s, about 12 m/s, about 15 m/s, about 20 m/s, or higher than 20 m/s. The targeted fluid velocity for fluid stream 210 can be any value within the range of aforementioned values. In some embodiments, at least two ports 230 are adapted to delivery respective fluid streams at different fluid velocities, for a given pressure of the fluid delivery mechanism. In some embodiments, at least two ports 230 are adapted to delivery respective fluid streams at the substantially the same fluid velocities, for a given pressure of the fluid delivery mechanism. In some embodiments, angular momentum is imparted to a thrombus by application of a) at least one fluid stream 210 that is directed at an oblique angle from a port 230, and/or b) at least two fluid streams 210 that have different fluid velocities. Advantageously, angular momentum produced in a thrombus may impart a (e g, centrifugal) force that assists in fragmentation and removal of the thrombus Advantageously, an increased cross-sectional area of the fluid lumen 145 reduces a required operating pressure of the fluid delivery mechanism to achieve a targeted fluid velocity of the fluid streams.

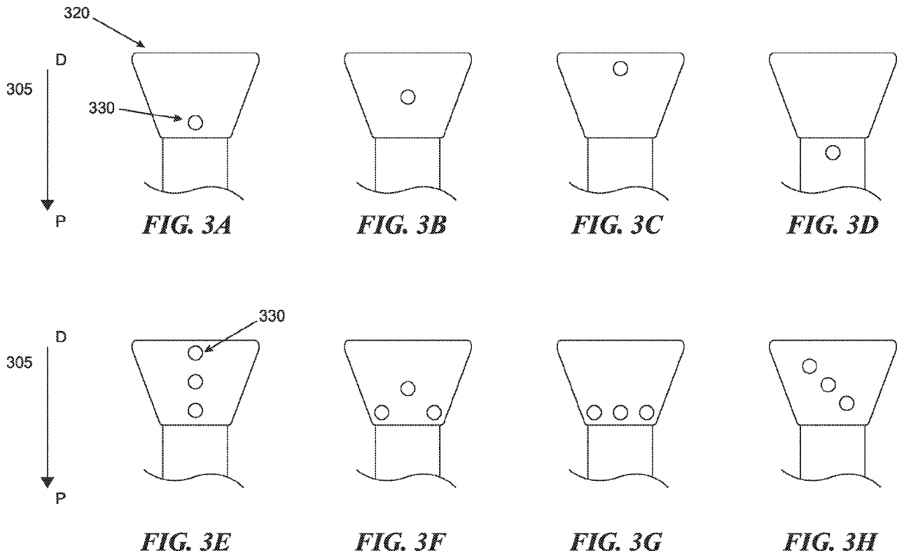

Referring to FIGS. 3A-3H, ports 330 can be arranged along various axial positions of the thrombus removal system. The thrombus removal system can include a flow axis 305 that is aligned with a general direction (e.g., distal-to-proximal) of flow for fluid that is aspirated therein. In some embodiments, a position of a port 330 comprises a) near a base of, b) in a middle portion of, c) in a distal portion of, or d) proximal to, a funnel portion 320 of the thrombus removal system. In some embodiments, at least two ports 330 are aligned along flow axis 305. In some embodiments, at least two ports 330 are arranged at a different axial positions along the flow axis 305. In some embodiments, at least two ports 330 are arranged (e.g., along a perimeter of the thrombus removal system) along a given axial position of the flow axis 305.

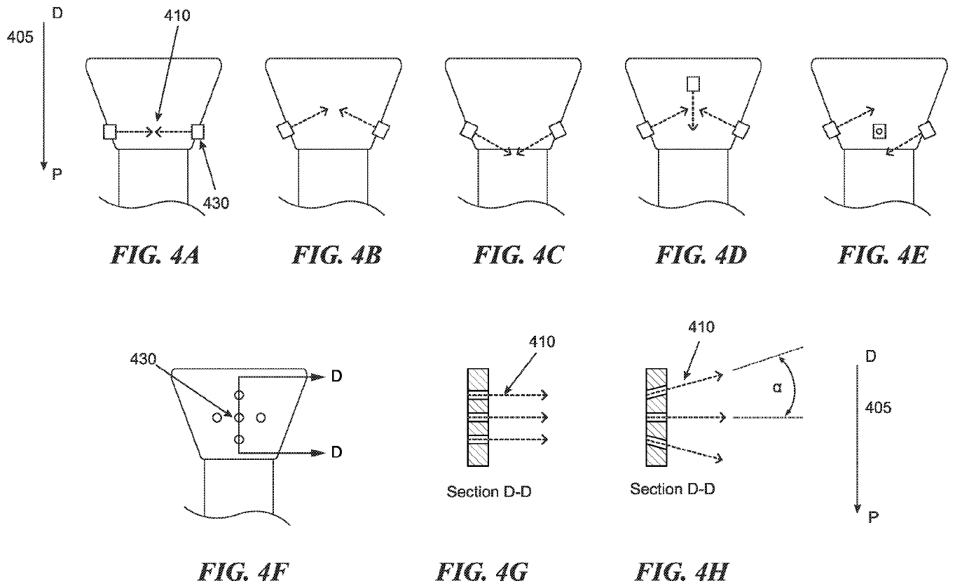

FIGS. 4A-4H depict various configurations of fluid streams 410 that are directed from respective ports 430. A fluid stream 410 can be directed along a path that is substantially orthogonal, proximal, and/or distal to the flow axis 405 (which is like to flow axis 305). In some embodiments, at least two fluid streams are directed in different directions with respect to the flow axis 405. In some embodiments, at least two fluid streams are directed in a same direction (e.g., proximally) with respect to the flow axis 405. In some embodiments, at least a first fluid stream is directed orthogonally, at least a second fluid stream is directed proximally, and at least a third fluid stream is directed distally with respect to the flow axis 405. An angle α may characterize an angle α fluid stream 410 is directed with respect to an axis that is orthogonal to the flow axis 405 (e.g., as shown in section D-D of FIGS. 4G and 4H). An intersection region of fluid streams can be within an interior portion of the thrombus removal system, and/or exterior (e.g., distal) to the thrombus removal system. In some embodiments, a fluid stream that is directed by a port 430 in a nominal direction (e.g., distally) is deflected along an altered path (e.g., proximally) by (e.g., suction) pressure generated by the aspiration mechanism during operation.

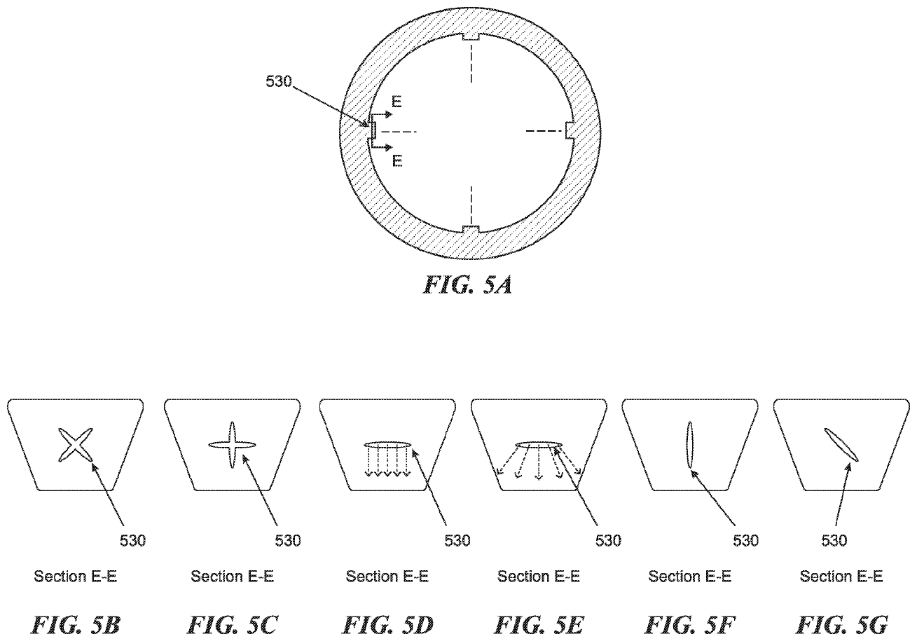

FIGS. 5A-5G illustrate a variety of exit aperture geometries with which ports 530 can be configured in accordance with embodiments of the present technology. Aperture geometries can comprise an oval, circular, cross (“x” shape), “t” shape, rectangle, or square shape. A fluid stream that is delivered from the port 530 can comprise substantially laminar flow (e.g., at the aperture), or a turbulent flow (e.g., that fans or outward).

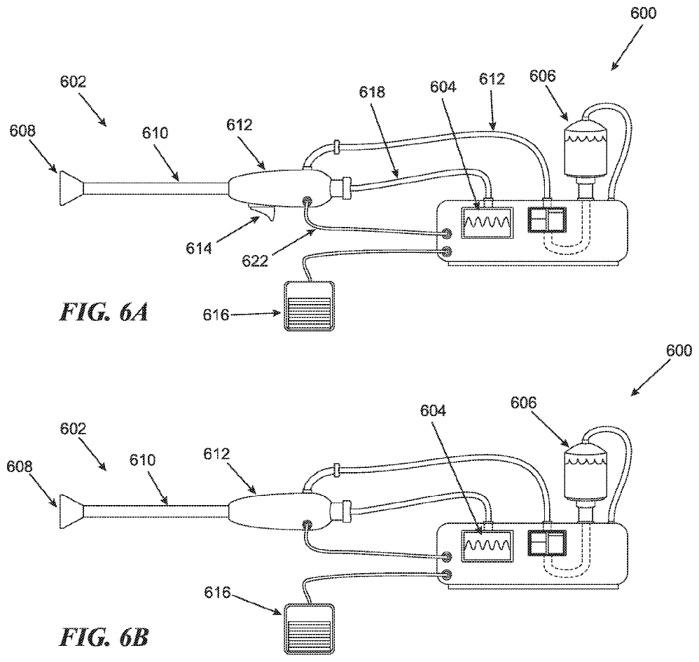

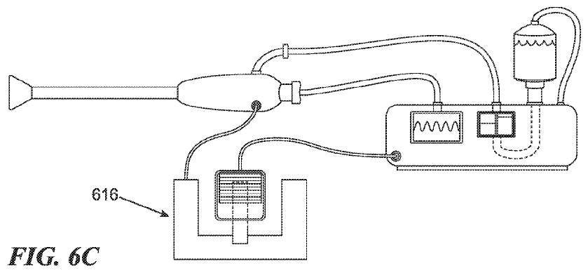

FIGS. 6A-6C illustrate various configurations of a thrombus removal system 600, including a thrombus removal device, 602, a vacuum source and cannister 604, and a fluid source 606. In some embodiments, the vacuum source and cannister and the fluid source are housed in a console unit that is detachably connected to the thrombus removal device. A fluid pump can be housed in the console, or alternatively, in the handle of the device. The console can include one or more CPUs, electronic controllers, or microcontrollers configured to control all functions of the system. The thrombus removal device 602 can include a funnel 608, a flexible shaft 610, a handle 612, and one or more controls 614 and 616. For example, in the embodiment shown in FIG. 6A, the device can include a finger switch or trigger 614 and a foot pedal or switch 616. These can be used to control aspiration and irrigation, respectively. Alternatively, as shown in the embodiment of FIG. 6B, the device can include only a foot switch 614, which can be used to control both functions, or in FIG. 6C, the device can include only an overpedal 616, also used to control both functions. It is also contemplated that an embodiment could include only a finger switch to control both aspiration and irrigation functions. As shown in FIG. 6A, the vacuum source can be coupled to the aspiration lumen of the device with a vacuum line 618. Any clots or other debris removed from a patient during therapy can be stored in the vacuum cannister 604. Similarly, the fluid source (e.g., a saline bag) can be coupled to the fluid lumens of the device with a fluid line 620.

Still referring to FIG. 6A, electronics line 622 can couple any electronics/sensors etc. from the device to the console/controllers of the system. The system console including the CPUs/electronic controllers can be configured to monitor fluid and pressure levels and adjust them automatically or in real-time as needed. In some embodiments, the CPUs/electronic controllers are configured to control the vacuum and irrigation as well as electromechanically stop and start both system in response to sensor data, such as pressure data, flow data, etc.

As is described above, aspiration occurs down the central lumen of the device and is provided by a vacuum pump in the console. The vacuum pump can include a container that collects any thrombus or debris removed from the patient.

### Steering and Navigation

As described above, the thrombus removal device can include steering mechanism(s) for navigating the device to a target treatment site or target thrombus. Steering can be performed in a variety of ways. As described above, one approach comprises one or more pull wires disposed in the auxiliary lumens as shown in FIGS. 1D, 1F, 1F, 1G, and 1H. Alternatively, the system can incorporate two concentric tubes with pushing and pulling the tubes relative to one another with each device having a preferential bend direction. Additionally, the device could be steered using ration with each device having a preferential bend.

To assist in steering and navigation, the systems and devices herein can further incorporate visual aids to allow real-time visualization of the thrombus removal device during a procedure. These visual aids can include fiducial markers embedded in the device including in the funnel, fluoroscopic dyes injected in or around the funnel, the device, the shaft, or features that result in echogenic regions within or around the device, including pockets of air within the funnel or device or small balloons that can be inflated to create echogenic regions under real-time imaging.

As one of skill in the art will appreciate from the disclosure herein, various components of the thrombus removal systems described above can be omitted without deviating from the scope of the present technology. As discussed previously, for example, the present technology can be used and/or modified to remove other types of emboli that may occlude a blood vessel, such as fat, tissue, or a foreign substance. Further, although some embodiments herein are described in the context of thrombus removal from a pulmonary artery, the disclosed technology may be applied to removal of thrombi and/or emboli from other portions of the vasculature (e.g., in neurovascular, coronary, or peripheral applications). Likewise, additional components not explicitly described above may be added to the thrombus removal systems without deviating from the scope of the present technology. Accordingly, the systems described herein are not limited to those configurations expressly identified, but rather encompasses variations and alterations of the described systems.

Dilator and/or Introducer Sheaths and Devices

The devices and systems described herein can further include introducer catheters or other tools designed and configured to assist with delivery of the thrombus removal system described herein to a target thrombus location. In some examples, the introducer catheter can include an introducer sheath that may optionally include a dilator or dilating component. For purposes of the disclosure herein, introducer catheters with a dilator or dilating component may be referred to herein as a “dilator sheath”. In some examples, this dilator sheath can be provided as a part of the introducer set. These dilator sheaths can be configured to access target blood vessels and include one or more lumens for advancement of the thrombus removal system to the target tissue site. The dilator sheath can further include one or more guidewire lumens to allow for advancement of a guidewire through the introducer set. The dilator sheath and thrombus removal system can then be passed over the guidewire into the blood vessel and to the target tissue site. In some embodiments, the guidewire comes installed on the dilator sheath. Embodiments are also provided in which the guidewire can be used to steer the dilator sheath in one or more directions.

The dilator sheaths disclosed herein can act to stretch a surgical opening in the skin and blood vessel to allow for the insertion of the sheath and thrombus removal device. When the dilator sheath is inserted into the blood vessel and advanced to the target tissue site, one or more ports or lumens in the dilator sheath allows a variety of catheter and/or medical devices, such as the thrombus removal system, to be advanced into the blood vessel at the target tissue site.

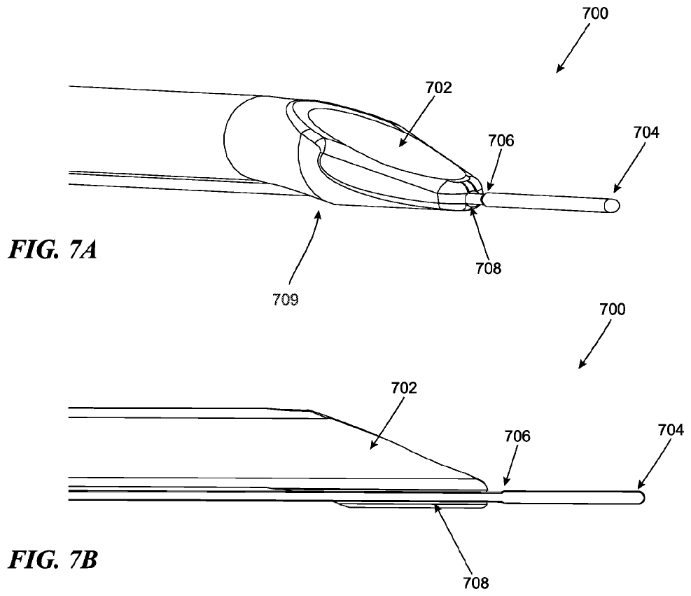

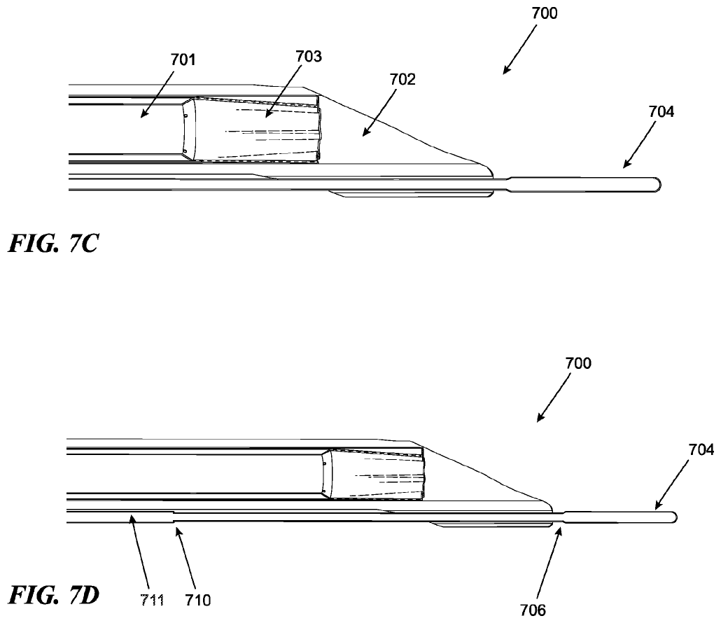

Referring to FIGS. 7A-7D, an embodiment of a dilator sheath 700 is provided which can include a device access lumen 702, a guidewire 704 having a distal step-up portion 706, and a guidewire lumen 708. As shown in FIGS. 7A-7D, the dilator sheath can have a flared dilator portion 709 configured to dilate or stretch the surgical opening to allow for insertion of the sheath. It should be understood that in other embodiments, the sheath may not have this flared dilator portion. In other embodiments, a separate dilator (not shown) can be inserted into the lumen of the sheath to serve the same function described above. In some embodiments, the guidewire lumen can extend approximately 1-10 cm along the length of the dilator sheath. As shown in FIGS. 7A-7D, the guidewire continues to extend proximally along the dilator sheath beyond the proximal end of the guidewire lumen. In another embodiment, instead of running proximally along the sheath, the guidewire lumen can feed into the device lumen 702, and the guidewire and return proximally through the device lumen 702.

FIG. 7B shows a cross-sectional view of the dilator sheath 700, including device access lumen 702 and guidewire lumen 708, and better shows the guidewire 704 and step-up portion 706 of the guidewire which has a larger outer diameter than the guidewire lumen to allow steering in one direction and backloading of the dilator sheath with the guidewire. For example, in one specific embodiment, the step-up portion 706 of the guidewire can have a diameter of approximately 0.035 cm, and the guidewire lumen can have a diameter of approximately 0.027 cm. Therefore, when the guidewire, and therefore the step-up 706, is pulled proximally into contact with the guidewire lumen, the dilator sheath is backloaded allowing for steering of the dilator sheath in one direction.

As mentioned above, the dilator sheath of the present disclosure integrates a dilator nosecone into the introducer catheter or sheath. Typically, the dilator device is separate from the introducer sheath, and the dilator is removed from the sheath after introduction. The described and illustrated embodiment integrates the dilator with the introducer sheath into a single unit.

In the embodiments of FIGS. 7C-7D, the dilator sheath 700 is shown with a medical device 701, such as the thrombus removal system described above, inserted into the device access lumen 702. As shown in this embodiment, the medical device 701 can include a collapsible funnel 703, which is compressed or constrained into a delivery configuration within the lumen 702 as shown. In some embodiments, a method of delivering a medical device 701 to a target tissue location can include: 1) advancing a guidewire into a lumen of a patient (e.g., a vessel); 2) loading the dilator sheath onto the guidewire via the guidewire lumen of the dilator sheath; 3) advancing the dilator sheath to the target tissue location over the guidewire; 4) during advancement, steering the dilator sheath by pulling proximally on the guidewire to backload the dilator sheath with an interference fit between the step-up of the guidewire and the guidewire lumen of the dilator sheath; and 5) loading the medical device into the lumen of the dilator sheath and advancing the medical device within the lumen to the target tissue site.

In the embodiment of FIG. 7D, the guidewire can include a distal step-up portion 706, as previously described, and also include a proximal step-up portion 710 located proximal to the guidewire lumen along the guidewire. The dual step-up configuration shown in FIG. 7D allows for interference between the guidewire and the guidewire lumen on both sides, providing the ability to steer in both directions by either backloading or frontloading the dilator catheter with the guidewire. For example, pulling back on the guidewire causes distal step-up portion 706 to engage with the guidewire lumen to allow for steering of the dilator sheath in one direction, and pushing forward on the guidewire causes proximal step-up portion 710 to engage with the guidewire lumen to allow for steering of the dilator sheath in another direction. In this embodiment, the guidewire can come pre-installed on the dilator sheath, since the proximal step-up portion would prevent loading the dilator sheath on the guidewire. However, in another embodiment, the proximal step-up portion 710 can optionally include an inflatable or expandable structure 711. For example, the proximal step-up portion 710 could include a balloon or other inflatable structure (inflatable with a medium such as with a gas or fluid) to enable steering in multiple directions while also being able to load the dilator sheath on the guidewire after guidewire insertion. In this example, a separate inflation lumen can be provided in the guidewire (not shown).

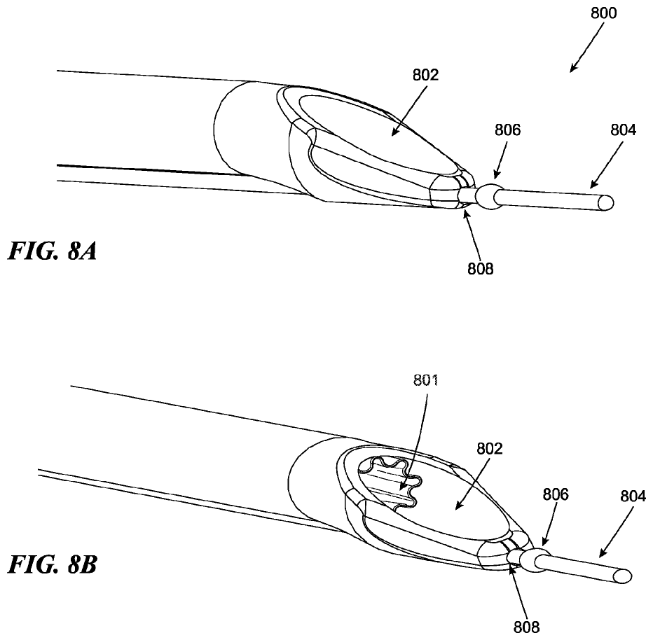

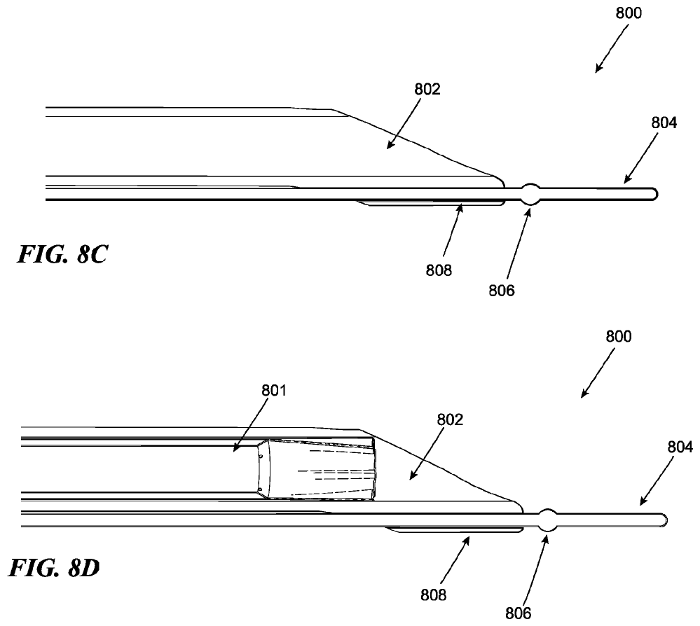

Referring to the embodiment of FIGS. 8A-8D, the dilator sheath 800 can include many of the same features as the dilator sheath described above, including a device access lumen 802, a guidewire 804, and a guidewire lumen 908. Additionally, the medical device 801 is shown in FIGS. 8B and 8D. However, in contrast to the embodiment of FIGS. 7A-7D in which the guidewire included one or more “step-ups”, the embodiment of FIGS. 8A-8D instead includes a protrusion 806 disposed on the guidewire distal to the guidewire lumen. As shown, the illustrated protrusion comprises a ball or spherical shape, but it should be understood that the protrusion can be other shapes. Preferably, the protrusion does not include any sharp or pointy edges to avoid catching on tissue. The protrusion shown in the embodiment of FIGS. 8A-8D serves the same functional purpose as the step-up portion on the guidewire described above; namely, the protrusion allows for backloading of the dilator sheath to allow for steering in one or more directions (depending on the number and location of protrusions).

While the protrusion of FIGS. 8A-8D is shown as being a permanent protrusion having a constant shape/diameter, in other embodiments the protrusion can comprise an inflatable or expandable member. For example, the guidewire could include an inflatable structure such as a balloon which could be inflated or expanded to allow for steerage of the dilator sheath. This inflatable or expandable structure can include either an inflation lumen or other expandable controls/connectors to enable expansion (not shown).

In other embodiments, a second protrusion can be positioned proximal to the guidewire lumen along the guidewire, similar to the dual step-up embodiment shown in FIG. 7D. As previously described, a permanent proximal or second protrusion allows for steering in multiple directions, but may require that the guidewire come pre-loaded on the dilator sheath. In some embodiments, the proximal or second protrusion can comprise an expandable structure such as a balloon or inflatable, to allow for steering in multiple directions but also allow for removal of the guidewire from the dilator sheath.

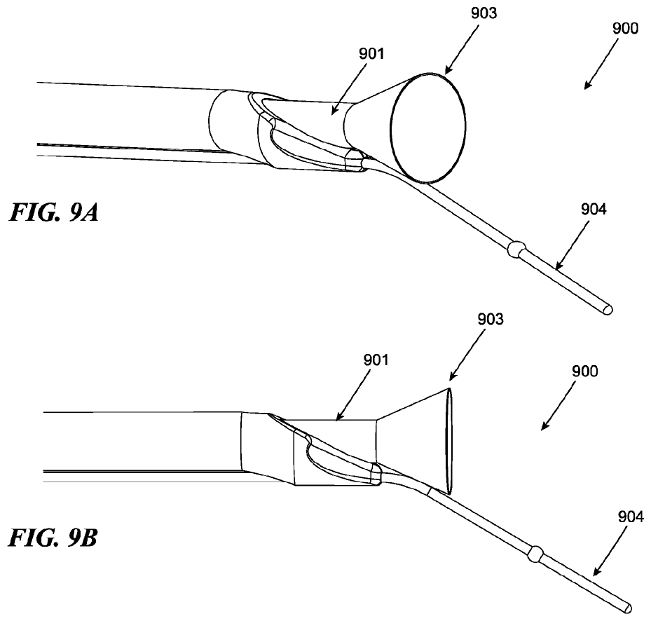

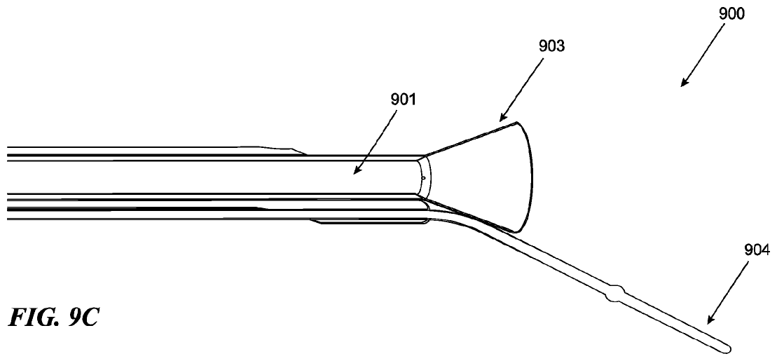

FIGS. 9A-9C show an embodiment in which the medical device 901 and collapsible funnel 903 have been advanced distally out of lumen 902 of the dilator sheath 900. FIG. 9B is a side view of the dilator sheath and medical device, and FIG. 9C is a cutaway view. When the device is advanced out of the sheath, the collapsible funnel 903 can automatically expand into a fully expanded configuration as shown. Accordingly, expansion of the funnel 903 can result in the guidewire 904 being pushed out of the way, as is shown in the figures.

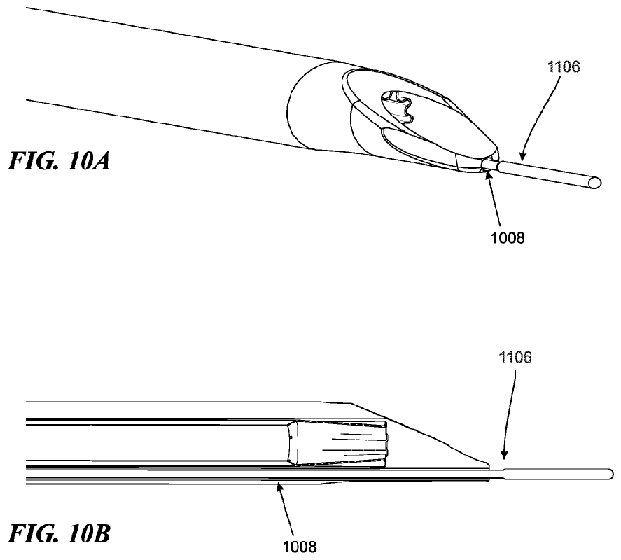

While the embodiments described above include a guidewire lumen disposed only near a distal end of the dilator sheath, the embodiment shown in FIGS. 10A-10B includes a full length or longer length guidewire lumen 1008. In some embodiments, the guidewire lumen can extend the full length of the sheath. The embodiment of FIGS. 10A-10B can further include a step-up portion or protrusion 1006 similar to as described above, to provide for backloading and/or steering of the dilator sheath by manipulating the interference fit between the guidewire step-up portion and the guidewire lumen.

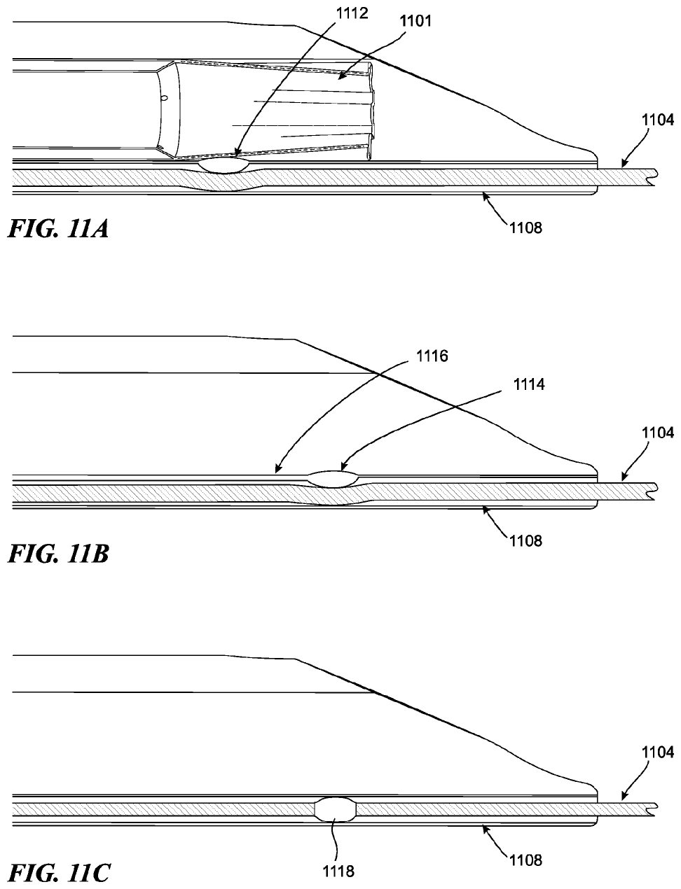

As described above, step-ups or protrusions along the guidewire can create an interference fit with the guidewire lumen to allow for steering of the dilator sheath. FIGS. 11A-11B illustrate another embodiment in which a depressible section 1112 in, near, or within the guidewire lumen 1108 can be expanded, closed, or clamped on or against the guidewire 1104 to allow for steering in more than one direction.

Referring to the embodiment of FIG. 11B, an expandable bladder 1114 can be introduced into the depressible section and expanded, creating interference with the guidewire in the guidewire lumen. This interference allows for steering of the dilator sheath in multiple directions by pulling or pushing on the guidewire. As shown in FIG. 11B, the expandable bladder 1114 can be fluidly coupled to an inflation lumen 1116 to allow for inflation of the expandable bladder. The inflation lumen can be coupled to a fluid source (not shown).

In the embodiment of FIG. 11C the depressible section 1112 and/or expandable bladder 1114 can be disposed on, in, or within the guidewire itself. In this example, the depressible section and/or expandable bladder is indicated by reference number 1118. As shown here, the depressible section and/or expandable bladder can be expanded to provide an interference fit between the guidewire and the guidewire lumen. Similar to the embodiment above, this interference fit can then be used to steer the dilator sheath by pulling or pushing on the guidewire, causing the dilator sheath to be steerable in at least two directions. In this embodiment, the interference can be applied anywhere along the guidewire lumen therefore the “steering point” can be set anywhere along the dilator catheter.

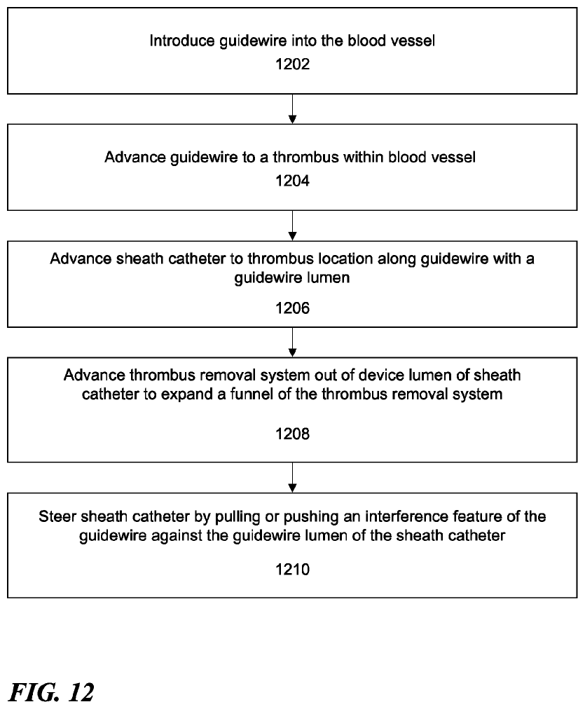

A flowchart is provided in FIG. 12 that describes one method of using the catheters, systems, and devices described herein. Referring to step 1202 of FIG. 12, a method can include introducing a guidewire into a lumen, such as a blood vessel. Next, at step 1204 of FIG. 12, the method can further include advancing the guidewire to a target location within the lumen, such as a thrombus within a blood vessel. Next, at step 1206 of FIG. 12, the method can include advancing a sheath catheter to the thrombus location along the guidewire with a guidewire lumen. In some embodiments, the sheath catheter can comprise a dilator sheath catheter. When the sheath catheter is in position, the method can further include, at step 1208, advancing a medical device, such as a thrombus removal system, out of a device lumen of the dilator sheath catheter. In the illustrated example, advancing a thrombus removal system out of the device lumen of the dilator sheath can allow for expansion of a funnel of the thrombus removal system to at least partially occlude the blood vessel. Finally, at step 1210 of FIG. 12, the method can include steering the dilator sheath catheter by pulling or pushing an interference feature of the guidewire against the guidewire lumen. As described above, the interference feature can comprise a step-up feature of the guidewire, can include a protrusion on the guidewire, or alternatively can comprise a depressible section in the guidewire lumen that can be collapsed or compressed, such as with an expandable bladder or balloon. Although step 1210 is described and illustrated as occurring after the other method steps, it should be understood that the sheath catheter can be steered at any point in the process. For example, the sheath catheter can be steered when the catheter is initially being guided over the guidewire towards the target location.

### EXAMPLES

Several aspects of the present technology are set forth in the following examples:

### CONCLUSION

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

A complete listing of the claims follows:

1. A method for steering an introducer catheter, the method comprising:
introducing a guidewire into a lumen, the guidewire including an interference feature on the guidewire;
advancing the guidewire towards a target location in the lumen;
inserting the guidewire into a guidewire lumen of the introducer catheter, wherein the guidewire lumen of the introducer catheter has a smaller diameter than the interference feature of the guidewire;
advancing the introducer catheter along the guidewire; and
engaging the interference feature of the guidewire against the guidewire lumen of the introducer catheter to steer the introducer catheter.

2. The method of claim 1, wherein the interference feature is disposed on a distal portion of the guidewire.

3. The method of claim 1, wherein the interference feature comprises a step-up portion in which a diameter of the guidewire increases to be larger than a guidewire lumen diameter of the introducer catheter.

4. The method of claim 1, wherein the interference feature comprises a protrusion having a larger diameter than a guidewire lumen diameter of the introducer catheter.

5. The method of claim 1, further comprising delivering a medical device to the target location via a lumen of the introducer catheter.

6. The method of claim 1, wherein the lumen comprises a blood vessel.

7. The method of claim 6, wherein the target location comprises a target thrombus location.

8. The method of claim 5, wherein the medical device comprises a thrombus removal device.

9. The method of claim 1, wherein the interference feature comprises an expandable member, wherein engaging the interference feature further comprises expanding the expandable member.

10. The method of claim 1, wherein engaging the interference feature further comprises pulling the guidewire proximally to engage the interference feature with the guidewire lumen.

11. The method of claim 1, wherein engaging the interference feature further comprises pushing the guidewire distally to engage the interference feature with the guidewire lumen.

12. An introducer catheter device, comprising:
an elongate shaft having a distal portion;
a device lumen configured to receive a medical device;
a guidewire lumen having a first diameter; and
a guidewire configured to be disposed within the guidewire lumen, the guidewire having a second diameter smaller than the first diameter, the guidewire also having at least one interference feature having a third diameter larger than the first diameter, the interference feature being configured to create an interference fit with the guidewire lumen when the guidewire is moved relative to the guidewire lumen for steering of the introducer catheter device in at least one direction.

13. The introducer catheter of claim 12, wherein the distal portion includes a dilator portion configured to stretch or expand a tissue.

14. The introducer catheter of claim 12, wherein the guidewire lumen extends along only the distal portion.

15. The introducer catheter of claim 12, wherein the guidewire lumen extends along the distal portion and the elongate shaft.

16. The introducer catheter of claim 12, wherein the interference feature comprises a step-up portion that includes a transition from the second diameter to the third diameter.

17. The introducer catheter of claim 12, wherein the interference feature comprises a protrusion.

18. The introducer catheter of claim 17, wherein the interference feature comprises a spherical shape.

19. The introducer catheter of claim 12, wherein the interference feature comprises an expandable structure.

20. The introducer catheter of claim 19, wherein the interference feature comprises a balloon.

21. The introducer catheter of claim 12, wherein the interference feature is positioned distal to the guidewire lumen when the guidewire is disposed within the guidewire lumen.

22. The introducer catheter of claim 21, wherein the interference feature is configured to provide an interference fit with the guidewire lumen when the guidewire is pulled proximally with respect to the introducer catheter.

23. The introducer catheter of claim 12, wherein the interference feature is positioned proximal to the guidewire lumen when the guidewire is disposed within the guidewire lumen.

24. The introducer catheter of claim 21, wherein the interference feature is configured to provide an interference fit with the guidewire lumen when the guidewire is pushed distally with respect to the introducer catheter.

25. The introducer catheter of claim 22, further comprising a second interference feature positioned proximal to the guidewire lumen when the guidewire is disposed within the guidewire lumen.

26. The introducer catheter of claim 25, wherein the second interference feature is configured to provide an interference fit with the guidewire lumen when the guidewire is pushed distally with respect to the introducer catheter.

27.-40. (canceled)
